(19) United States
(12) Patent Application Publication
(10) Pub. No.: US 2009/0227827 A1
(43) Pub. Date: Sep. 10, 2009

(54) Vaginal applicator for brachytherapy treatment of vaginal and endometrial disease

(76) Inventors: Heike Hausen, Redwood City, CA (US); Thomas W. Rusch, Hopkins, MN (US); James E. Jervis, Atherton, CA (US)

Correspondence Address: Thomas M. Freiburger, P.O. Box 1026, Tiburon, CA, 94920, US

(21) Appl. No.: 12/075,120

(22) Filed: Mar. 5, 2008

(51) Int. Cl. A61M 36/10

(52) U.S. Cl. 600/6; 600/7

(57) ABSTRACT

A vaginal brachytherapy applicator has an insertable tip section, preferably rigid or at least firm enough to retain its shape when inserted, and a tubular handle section extending proximally from the tip section. A common lumen extends from the handle into and through most of the length of the tip section. In one embodiment the tip section is made of a flexible open-celled foam material with a substantially impermeable outer skin, and in this case the tip section can be contracted, or stretched lengthwise to reduce its diameter, for ease of insertion, then re-expanded to its nominal and working configuration prior to insertion of a radiation source into the lumen and irradiation of tissue surrounding the tip section.

### BACKGROUND OF THE INVENTION

This invention relates to brachytherapy treatment of gynecological proliferative disease as adjuvant care following surgery. More particularly, it pertains to intracavitary application of therapeutic x-ray radiation emitted from miniature x-ray tubes from within the vagina or rectum of the patient.

It has been demonstrated in many areas of surgical oncology that adjuvant radiation treatment following tumor resection reduces the likelihood of recurrence of cancer or other proliferative disease. It has also been shown that brachytherapy is as effective as external beam therapy, and furthermore, that quality-of-life issues are superior after brachytherapy. It is therefore desirable that brachytherapy be made available to as great a population of patients as possible.

In early brachytherapy applications, fluid media comprising radioisotopes were used to fill a balloon positioned within a body cavity or organ in order to provide therapeutic radiation. Later, it was recognized that spacing the radiation source away from the tissues being treated provided means to deliver the prescribed radiation with reduced likelihood of overdosing normal tissue. This led to filling the balloon with an attenuating medium, often saline solution, and addition of source guides within the balloon in order to position solid isotope sources with some accuracy. The traditional sources are isotopic seeds, often of iridium 192 positioned on wires, which are manipulated within the source guides to deliver the prescribed treatment to the target tissue surrounding the balloon and the treatment cavity.

Treatment is often delivered in fractions spaced in time to take advantage of the fact that normal cells recover from radiation exposure whereas diseased cells do not.

The devices inserted into the body and used to create and manage the spacing between the target tissue and radiation source are usually called applicators. Applicators typically used for delivery of radiotherapy from within the vagina, or alternatively the rectum or the adjacent section of bowel are tubular cylinders of materials which allow transmission of radiation, and control the necessary spacing. U.S. Pat. No. 7,338,430 describes a balloon applicator and copending application Ser. No. 11/811,069 discloses an everting applicator, and Ser. No. 11/805,065 describes a form-fitted applicator, all for gynecological brachytherapy.

Because the isotopes frequently used for brachytherapy often emit high-energy radiation components which can penetrate deeply into the materials to which they are exposed, as well as because they emit continuously, they can only be used in special, heavily-shielded rooms. In addition, concerns for the safety of personnel require isolation of the patient during treatment, shielded storage at all other times, and automated handling between the storage chamber and the applicator implanted in the patient. In total, the capital expense required for such facilities dictates that treatment centers be located in urban areas so as to serve sizeable patient populations. This can result in under serving rural patients who cannot repeatedly travel to urban treatment centers for radiation treatment. Furthermore, patient isolation during treatment is inconvenient for therapists, not to mention daunting for the patients under treatment. With such brachytherapy, it is clear that improvements are needed in several respects.

Recently, miniature electronic x-ray tubes have provided a preferable alternative to use of isotopes. Such tubes do not emit continuously, they only emit when powered in a manner causing emission and they can be turned on and off, or if desired, modulated such that their penetration depth can be controlled (by control of acceleration voltage) and their dose intensity can be controlled (by filament current) as well. Electronic brachytherapy sources are therefore more versatile, and can accommodate a wide variety of prescription detail. Isotope sources cannot be controlled in this manner. Furthermore, the x-ray energy spectrum in ranges suitable for brachytherapy permits the therapist to be in the room with the patient, and any need for heavily shielded structures, or “bunkers” is eliminated. Therapy can therefore proceed in almost any medical facility, urban, rural or even mobile. With miniature x-ray tubes, a much greater population of patients can therefore be served, and the costs of therapy are greatly reduced, and patient isolation is not required.

The solid applicators of the prior art can be difficult to insert into the vagina, rectum or bowel, and can result in patient discomfort. Embodiments of this invention address applicator improvements which, when used with electronic x-ray sources, improve radiation dosimetry as well as patient comfort. It should be noted that the discussion of this invention herein is largely in terms of vaginal application; however, it is to be understood that similar therapy for cancer of the rectum or bowel are to be considered within the scope of the invention.

Objectives of this invention are to help make brachytherapy practical in almost any medical facility by eliminating the need for bunkers and shielded isotope storage, thus significantly reducing capital costs of treatment facilities, eliminating the need for patient isolation during treatment, and improving dosimetry of the radiotherapy. Other objectives will become apparent to those of skill in the art from the following drawings and description.

### SUMMARY OF THE INVENTION

One preferred embodiment of the invention comprises a two-part applicator in which a handle is fastened to a tip section shaped for insertion into the vagina, leaving at least the proximal end of the handle protruding outside the patient's body. Both parts are substantially rigid; the handle is preferably of metal, such as stainless steel, and the tip of an engineering plastic, such as NORYL (GE Plastics Division; Pittsfield, Mass.). NORYL can be filled with image enhancing fillers like barium sulfate, for example, to assist the medical practitioner to determine proper placement within the vagina using conventional x-ray or CT techniques, and its properties allow autoclaving. In addition, its radiation attenuation properties in the energy range consistent with the use of miniature x-ray tubes are substantially similar to that of tissue, making treatment planning relatively easy. If desired, the handle portion of the applicator may also be of engineering plastic. A conventional connection method can be used to join one part to the other, for example the joint may be threaded, bonded or clamped.

Both the handle and tip section are cored along their lengths such that, when assembled, they cooperatively form a lumen leading from outside the patient through the handle and into the tip section for placement of a radiation source during treatment, and optionally for manipulation of the source in order to deliver a prescription dose to the vaginal tissues. With miniature x-ray tubes, the distribution of the radiation emission intensities, normally described in terms of isodose surfaces, can be shaped by appropriate tube design, particularly anode design. Such methods are well understood by those of skill in the art, and are also discussed in the technical literature (see below).

The cumulative absorbed dose to be delivered to the patient needs to conform to the patient's prescription. Most prescription radiotherapy is delivered in a series of fractions separated in time, each fraction comprising a series of emission periods delivered from a sequence of source positions located within the applicator. While the shape of the applicator needs to generally match the anatomy needing treatment, it is also generally preferred that the external shape (and therefore substantially the attenuation) of the distal end of the tip section and the isodose patterns of the radiation source be similar, and therefore result in uniform dose intensity (cumulatively) at the applicator surface. In addition, proper insertion of an applicator includes applying sufficient (but gentle) force to the applicator in order to shape the vaginal cavity to the applicator shape. Such an applicator so positioned will provide substantially uniform radiation intensity incident on the vaginal tissues adjacent to the applicator, and a prescription dose can therefore be delivered to the tissues at a uniform prescription depth—often about five (5) millimeters. Following the treatment, both the source and applicator are removed from the vagina.

If desired, other applicator shapes and coring can be used to create other isodose shapes responsive to individual patient requirements and prescriptions. Alternatively, a standard variety of shapes and sizes can be manufactured to accommodate a variety of common patient circumstances by appropriate selection.

Miniature x-ray tubes which emit substantially isotropically, or optionally tubes with emissions limited to a predetermined solid angle, can be used advantageously to shape the radiation dose distribution delivered to the patient. As mentioned above, these sources additionally have the capability of being modulated or even switched on and off. Such x-ray tubes are discussed in Atoms, Radiation and Radiation Protection, Second Edition, John E. Turner, Ph.D., CHP, 1995, John Wiley & Sons, Section 2.10. Such solid-angle directional source emissions can also be produced by selective shielding of isotropic x-ray sources following the methods described in application Ser. No. 11/471,277, incorporated herein in its entirety by reference, and in fact, the solid angle parameters of the source can be varied as well. Shielding methods can also be used to limit emissions of either x-ray tubes or isotopes, thus producing similar patterns to the directional emission patterns of x-ray sources as described above. Isotope sources cannot in principle be modulated, however.

Another preferred embodiment of the present invention preferably comprises a different applicator tip section. The first part remains a rigid tubular handle which may be of metal or engineering plastic. The second part or tip section of this embodiment comprises a skin-covered sponge, that is to say, a resilient, substantially cylindrical probe, preferably of open-cell foam, with an impermeable skin on the outside thereof. Again, it is desired that the skin be doped with barium sulfate as described above to facilitate imaging. The two parts are conventionally joined, for example by bonding, and both parts are cored as described previously, creating a lumen to permit positioning a radiation source within the applicator, and if desired, manipulated in accordance with the patient's treatment plan.

The coring can, optionally, further comprise a fluid lumen leading from outside the patient to the open-cell foam portion at the distal end of the applicator. Because the foam has a skin, vacuum can be applied to the central lumen and this will collapse or tend to collapse the foam into a more compact form, reducing the diameter of the tip section and facilitating insertion of the applicator into the vagina. Depending on the foam and skin material(s) chosen, wetting the foam, for example with saline, may facilitate collapse of the tip section. When collapsed, the applicator is inserted into the vagina, foam end first, such that the vagina is substantially filled by the foam end, leaving at least the proximal end of the handle exposed outside the patient.

Once the applicator is inserted, the vacuum is released, and if the recovery force of the foam does not fill the vagina satisfactorily, fluid pressure may be applied through the lumen to assist expansion. When the vagina is filled satisfactorily, the radiation source, preferably a miniature x-ray tube, is inserted into the applicator lumen from without the patient, and therapy is commenced. Following the treatment, both the source and applicator are removed from the vagina, and optionally sterilized for reuse as necessary.

The shape-change feature provides a benefit in that during applicator insertion, the diameter of the applicator is reduced as it is passed through the introitus leading to the vagina, thus producing less discomfort during applicator placement.

The handle and foam portion of the applicator can be separable, facilitating the combination of a reusable handle combined with a disposable foam portion. The fastening method therebetween can be conventional, for example coaxial portions where the outer foam sleeve is clamped onto a tubular extension of the handle. If desired, rather than use of the central lumen for application of vacuum, the fluid lumen leading to the foam can be a free standing tube secured to the foam and positioned alongside the handle during applicator insertion and therapy, and this lumen can be used for vacuum application.

The foam portion can be shaped to suit an individual patient's anatomy and prescription, or a standard variety of shapes and sizes can be manufactured to accommodate a variety of patient circumstances by appropriate selection.

Other elements can be built into the resilient portion of the applicator to similarly reduce the diameter of the applicator to ease insertion into the vagina. Examples include inserting a rigid wand into the source lumen and pushing against the distal end to lengthen the resilient section, reducing its diameter. Such distortion of the resilient portion may be facilitated by embedding a braided section comprising plastic strands, for example of polyester, which can be lengthened and reduced in diameter as described above, or shortened and increased in diameter be pulling on the distal end of the source lumen. With a foam material having a strong shape-return property, pulling may not be needed. A threaded connection at the distal tip of the central lumen and wand can serve to provide the pulling tension. If the properties of the resilient section are such that an effective female thread cannot be formed to mate with the male thread on the wand, a structural insert with the female thread may be bonded into the distal tip of the resilient tip lumen for tensile strength purposes.

Different combinations of elements described above can be combined in other permutations as will be apparent to those of skill in the art without departing from the scope of the invention.

### BRIEF DESCRIPTION OF THE DRAWINGS

FIG. 1A is a longitudinal section view of an applicator of the invention.

FIG. 1B is an applicator similar to that of FIG. 1A, but with attenuating masks positioned in the tip section of the applicator.

FIG. 1C is a transverse section through the tip section of the applicator of FIG. 1B.

FIG. 2 is an alternative distal tip shape which may be utilized in some therapeutic situations.

FIG. 3 is a longitudinal section view of an alternate applicator embodiment.

FIG. 4A is a longitudinal schematic view of the applicator of FIG. 3 prepared for insertion into the vaginal cavity of the patient.

FIG. 4B shows the applicator of FIG. 4A expanded within the vaginal cavity of the patient.

### DESCRIPTION OF PREFERRED EMBODIMENTS

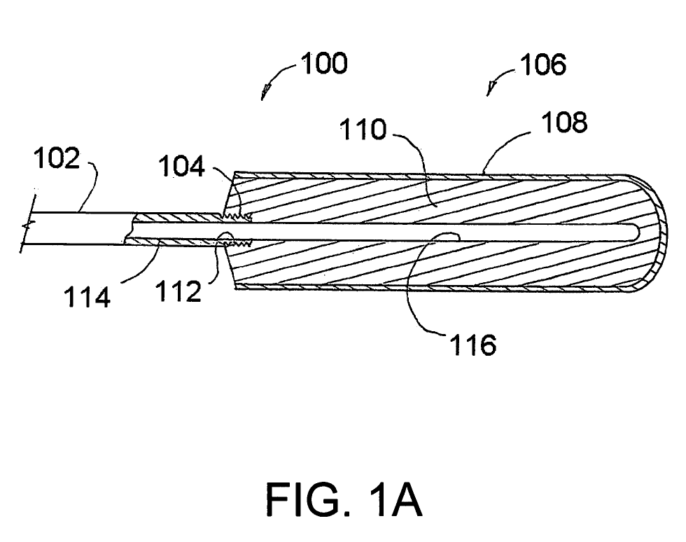

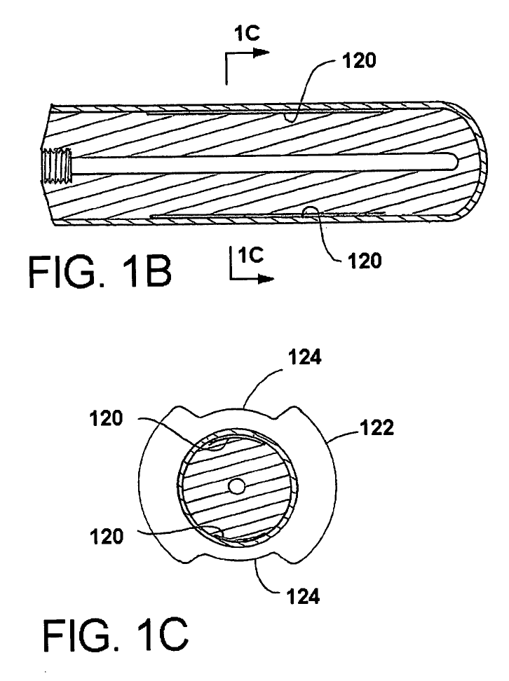

FIG. 1A shows a preferred applicator 100 embodiment of the invention. A metal handle 102, preferably of Stainless Steel, is joined to a molded plastic tip section 106, shaped to be inserted into and fill the vaginal cavity of the patient. The handle 102 alternatively can be a hard plastic material. If desired, the tip section can be sized and shaped to dilate the vagina to some degree, urging the tissue into conformance with the shape of the applicator. Preferably the tip section comprises an outer skin 108 which has a smooth exterior surface, and is doped with an x-ray attenuating material, for example barium sulfate, to facilitate imaging of the applicator outer surface by conventional x-ray or CT scanning methods. A filler material 110 within the tip section outer skin comprises a solid polymer having attenuation properties which, in concert with the attenuation of the said skin, results in collective attenuation substantially the same as the attenuation of a similar volume of saline solution. Saline solution in turn has attenuation similar to the attenuation of tissue, and attenuation matching simplifies the treatment planning process for the therapist. One material useable for both the skin and the filler is NORYL (GE Plastics; Pittsfield, Mass.). When doped with barium sulfate, the attenuation properties of the skin increase above that of saline making the skin appear during conventional x-ray or CT imaging; when undoped, the balance of the tip section has attenuation slightly below that of saline. Collectively, the result is similar to saline and does not disturb conventional treatment planning. Another construction similar to saline is a doped skin of RADEL (Solvay Advanced Polymers; Alpharetta, Ga.), and a filler of ULTEM (GE Plastics). In one form the skin can simply be the outer surface of the core.

The imaging mentioned above serves to verify correct applicator placement within the patient's vaginal cavity. Imaging can also be used to check tissue conformance between the treatment cavity interior surface and the applicator exterior surface preparatory to initiation of radiation treatment.

Referring again to FIG. 1A, the tubular handle 102 may have features at its proximal end (not shown) to cooperate with automated handling apparatus comprising part of a complete therapeutic treatment system (not shown). At its distal end, the handle comprises a male thread 104 to cooperate with a similar female thread 112 at the proximal end of the tip section 106 for the purpose of joining the two elements. Other conventional fastening methods can be used, including bonding. A threaded connection facilitates sterilization and reuse of the handle. The tip section can be sterilized and reused, sterilized for same-patient reuse, then discarded, or it can be one-time-use disposable.

The central lumen 114 in the tubular handle connects co-axially with a central lumen 116 in the tip section, the two used collectively for the purpose of advancing a radiation source into the vagina within the applicator, and stepping or manipulating it in accordance with the patient's treatment plan. A preferred shape of the tip section 106 is substantially cylindrical, with the shape of the tip section mimicking the cumulative isodose patterns of the source when stepped. As described above, when the distal end 117 is configured generally partial-spherically, or dome-shaped, the dose intensity around the tip is uniform, and therefore the absorbed dose at prescription depth surrounding the vaginal vault is substantially uniform as well. If a non-uniform dose intensity is desired in some clinical circumstances, either or both the isodose pattern of the x-ray source and the shape of the distal end 117 of the tip section can be varied to produce the desired isodose shape. As stated above, anode design is typically used to conform emission patterns to desired shapes. The lumen 116 for the radiation source can also be moved off center, or more than one lumen can be employed within the applicator to effect an irregular isodose pattern.

One particularly useful, non-isotropic shape is one where the dose intensity directed toward the bladder (anteriorly) and rectum and bowel (posteriorly) of the patient is moderated relative to the overall dose intensity. Any overdose to these tissue structures can damage normal tissue and perhaps function. Such a pattern can be created by partial shielding of the emissions, for example, by utilizing the principles disclosed in co-pending applications Ser. Nos. 11/471,013 and 11/471,277, filed Jun. 19, 2006, both incorporated herein in their entireties by reference. By applying silver (or other) masks lengthwise at 180° along all or a portion of the length of the tip section, and positioning them adjacent to the bladder and bowel, attenuation is locally increased, protecting that anatomy. These masks 120 are shown in longitudinal section in FIG. 1B, and in transverse section in FIG. 1C. FIG. 1C also shows an exemplary isodose pattern 122, illustrating the lower dose intensity regions 124 which are oriented to at least partially shadow the bladder and bowel. Other mask materials, shapes and positioning can be used to create other desireable isodose patterns, including by methods and apparatus such as those described (as shielding) in copending application Ser. No. 11/471,277. Masks can be positioned on the exterior of the outer skin if desired, with proper protection. The use of such shielding has the advantage of allowing use of a substantially isotropic source which is likely to be preferred in other treatment areas. More generally, the teachings of the Turner reference (see Summary above), conventional shielding, x-ray tube anode practice, and distal applicator shapes and properties, can all be used to create custom or specialty isodose patterns as is familiar to those of skill in the art. Furthermore, rather than positioning the masks near the periphery of the applicator tip section, the masks can be positioned nearer the center of the applicator, such as on the source catheter outer surface.

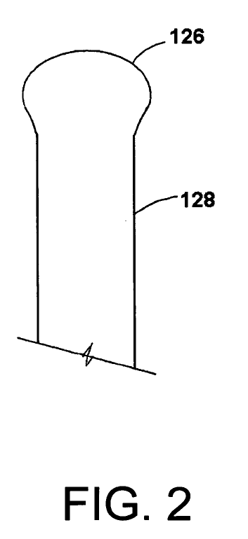

FIG. 2 depicts a shape variation in the distal end of the tip section of an alternate applicator 125, comprising a bulb 126 blended into the cylindrical body 128 of the tip section. The purpose of this alternate shape is to conform to anatomy variations found in some patients. As with configuring the isodose patterns to the applicator tip shape shown in FIG. 1A, appropriate x-ray tube design adjustments can be made to produce isodose patterns which resemble this shape.

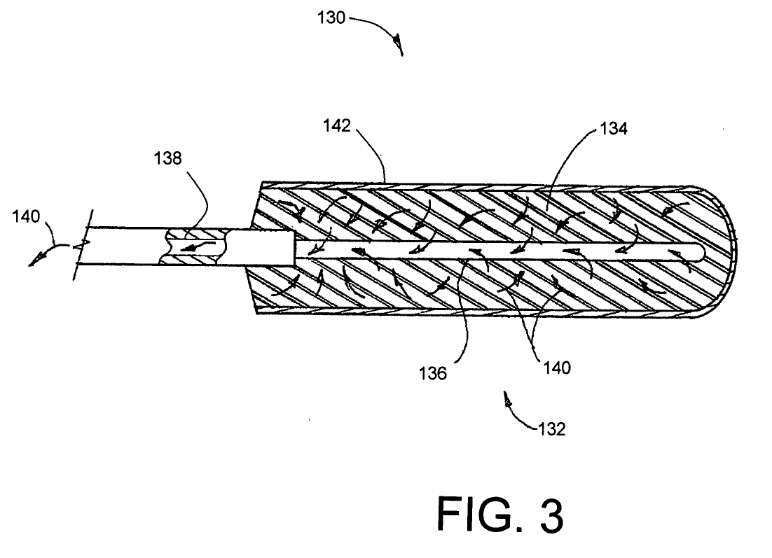

FIG. 3 shows another preferred embodiment 130 where all or part of the tip section 132 can be reduced in diameter during insertion into the vagina, and is then allowed to return to its desired shape before imaging and initiation of radiation emissions. The portion of the tip section 132 which is capable of diameter reduction is a skin-covered sponge-like structure, cylindrical in form, comprising a molded open-cell resilient foam core 134, in communication with a central lumen 136 which in turn is in communication with the handle lumen 138 as above. The handle and tip section are preferably bonded together using conventional methods and materials. The lumen 136 is also in communication with a vacuum source, typically wall suction in the treatment room, and connected through a proximal hub (not shown) mounted or in communication with the handle lumen 138 from outside the patient. An example where only a portion of the tip section is capable of diameter reduction is the bulb 126 of FIG. 2. The cylindrical portion 128 of the applicator of FIG. 2 could be solid if desired. Alternatively, the entirety of the tip section shown in FIG. 2 can be capable of diameter reduction.

The core is preferably of silicone rubber, and the connected voids of the open cell structure of the foam core 134 are in fluid communication with the lumina 136 and 138 (which form a continuous lumen). Such a structure can be created within a heated mold containing the silicone polymer and a blowing agent such as sodium bicarbonate. The choice of blowing agent preferably is made such that the degradation temperature of the agent is similar to or substantially the same as the temperature at which the polymer viscosity is near or at its minimum. The tip section 132 further comprises a substantially impervious skin 142 over the foam core, such that the outer shape of the tip section is as desired to fill and/or shape the vagina. The skin 142 is thin and can be formed by dipping the core 134 into silicone rubber and curing, for example. Polyurethane is an alternate material which is appropriate for either the foamed core, the dipped skin or both. Other blowing agents are azodicarbonamide and 2,2-azobisisobutyronitrile. Again, as mentioned in the Summary above, with the choice of a foam material which is softened by water, the tip section could be wetted with saline to facilitate its collapse. Such a foam might be of cellulose, and the dipped skin of silicone or polyurethane as previously described. Procedures of this sort are well known to those of skill in the art.

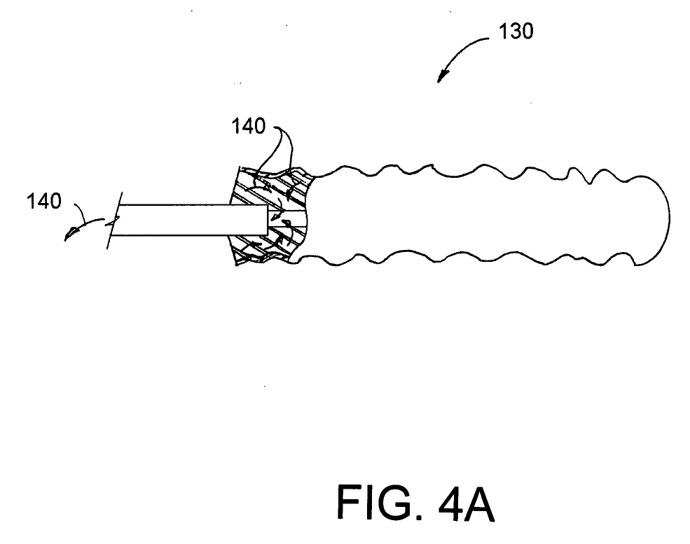

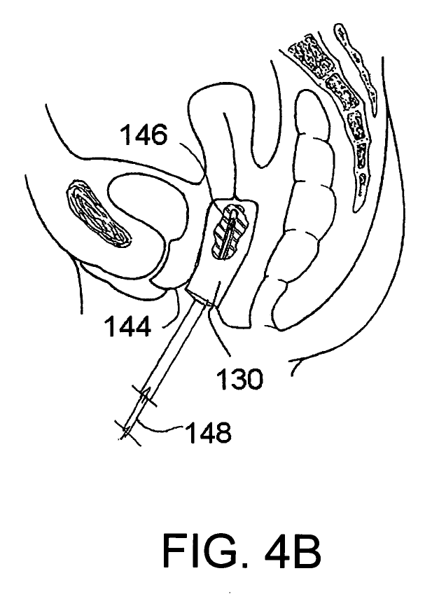

When the central lumina 136 and 138 are connected to vacuum but otherwise sealed, the foam core 134 will contract more or less randomly, or tend to contract around the central lumen, pulling the skin 142 inward as well. See FIG. 4A, discussed below. Arrows 140 are shown in FIG. 3 generally to depict the fluid flow upon such application of vacuum. If necessary, a wand (not shown) can be inserted into the central lumina to provide stability to the tip section during insertion into the vagina, and then removed and replaced by the radiation source on its catheter (not shown). In this configuration the applicator will be easier to insert and position within the vagina. Releasing the vacuum with the applicator properly positioned will allow the resilient tip section 140 to recover its original diameter, filling the vagina.

As noted above it is preferred that the attenuation properties of the tip section approximate saline, and it is also desirable that the applicator periphery be doped to facilitate imaging. To help approximate saline, rather than releasing the vacuum to the atmosphere, the foam may be connected to a supply of saline such that as the resilient foam 134 of the tip section 132 expands back to normal size, it will imbibe saline, filling its open-cell structure. To speed return to normal size, the saline supply may be pressurized if desired. Radio-opacity to enhance imaging can again result from doping of the exemplary silicone material into which the foam core is dipped. An exemplary dopant is barium sulfate as previously mentioned. Alternatively, the outer surface of the foam core 134 can be surface treated with an attenuating material before applying the skin. Measures to create non-isotropic dose distributions similar to those described in connection with the applicator of FIG. 1 may be employed with the foam applicator embodiment 130 of FIG. 3 similarly.

FIG. 4A shows the applicator 130 of FIG. 3 in a state where its size has been randomly reduced by application of vacuum (see arrows 140). FIG. 4B shows the same applicator positioned within the vagina 144 and having returned to its normal or expanded size, which may be by back-filling with saline as described earlier, under pressure if necessary either to speed its expansion or for therapeutic purposes. A radiation source 146 is mounted on the end of a source catheter 148, and positioned in the central lumen of the applicator 130 in the vagina 144.

As explained above, alternate, non-vacuum method of inserting the foam applicator described with respect to FIG. 3 comprises using a long, solid (metal or engineering polymer) wand (not shown) within the central lumen 138 to stretch the foam distal section axially, thus reducing its diameter, before advancing the tip section into the vagina. After tip section insertion into the vagina, the wand is held in a constant position with respect to the anatomy as the handle of the applicator is advanced toward the vagina. As it is advanced, the tip section diameter will increase until the vagina is filled, after which the wand can be removed and the radiation source and catheter inserted.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A gynecological brachytherapy applicator comprising:
a handle including a proximal end to extend out of a patient when the applicator is in use,
a tip section secured to the handle and extending distally from the handle,
a lumen extending through the handle and into and through most of the length of the tip section, and
the tip section comprising an outer skin and a core within the skin and surrounding the lumen, the core and outer skin together approximating the attenuating properties of human tissue.

2. An applicator as in claim 1, wherein the handle is of substantially smaller diameter than the tip section and is substantially rigid, and the tip section being retained to the handle by screw threads.

3. An applicator as in claim 2, wherein the core comprises a rigid polymer.

4. An applicator as in claim 1, wherein the core is of the material NORYL.

5. An applicator as in claim 1, wherein the pin outer skin of the top section includes a radiation attenuating material so as to be visible in an x-ray or CT scan taken from outside the patient.

6. An applicator as in claim 1, wherein the tip section is substantially cylindrical in shape, with a distal end having generally a dome shape.

7. An applicator as in claim 1, wherein the core comprises an essentially sponge-like material of open-cell resilient foam, whereby the tip section is capable of diameter reduction by application of vacuum to the lumen for ease of insertion into the patient, with the outer skin being smooth and substantially impermeable.

8. An applicator as in claim 7, wherein the tip section is substantially cylindrical in shape, with a distal end having generally a dome shape.

9. An applicator as in claim 7, wherein the open-cell foam material of the core is silicone rubber.

10. An applicator as in claim 9, wherein the outer skin is formed of silicone rubber, applied wet to the exterior of the foam core and cured.

11. An applicator as in claim 1, wherein the outer skin is smooth and of polyurethane.

12. An applicator as in claim 1, wherein the core is of resilient polyurethane foam.

13. An applicator as in claim 1, further including at least one radiation attenuating mask on the tip section, positioned to protect sensitive tissues of the patient.

14. A method for inserting and properly positioning a brachytherapy applicator in a cavity of a patient for administering brachytherapy radiation, comprising:
providing an applicator having a handle and a tip section connected to the handle and extending distally of the handle, with a lumen extending through the handle and into and through most of the length of the tip section,
the tip section comprising an outer skin and a resilient core within the skin and surrounding the lumen,
applying vacuum to the core of the tip section to withdraw air from the core and thus to partially collapse and reduce the diameter of the tip section,
with the tip section in a reduced-diameter condition, inserting the applicator into a cavity of a patient to be treated,
releasing vacuum from the core of the tip section and causing the core to re-expand and to assume a fully-expanded, normal configuration within the patient, and
placing a radiation source within the lumen, inside the tip section, and irradiating tissue surrounding the tip section in accordance with a treatment plan.

15. The method of claim 14, wherein the application of vacuum to the core comprises applying vacuum to the lumen, which communicates with the core within the tip section.

16. The method of claim 14, wherein the core comprises an open-celled foam material.

17. The method of claim 14, wherein the outer skin is substantially impermeable.

18. The method of claim 14, wherein the core and outer skin together approximate the attenuating properties of human tissue.

19. The method of claim 14, wherein the core comprises open-celled foam of silicone rubber material.

20. The method of claim 14, wherein the core comprises an open-celled foam of cellulose material.

21. The method of claim 14, wherein the tip section is rigidly bonded to the handle.

22. The method of claim 14, wherein the outer skin is smooth and comprises a different material from the core.

23. The method of claim 14, further including imaging the position of the applicator after inserting the applicator into the cavity of the patient, from outside the patient to confirm correct positioning of the applicator in the cavity prior to irradiating the tissue.

24. The method of claim 14, wherein the cavity of the patient comprises a vagina.

25. The method of claim 14, further including, prior to inserting the applicator, placing one or more attenuating masks on the tip section, positioned to protect sensitive tissues of the patient near the cavity.

26. A method for inserting and properly positioning a gynecological brachytherapy applicator in a cavity of a patient for administering brachytherapy radiation, comprising:
providing an applicator having a handle and a tip section connected to the handle and extending distally of the handle, with a lumen extending through the handle and into and through most of the length of the tip section,
the tip section comprising an outer skin and a resilient core within the skin and surrounding the lumen,
pushing a rigid wand through the lumen, to the end of the lumen in the tip section, to extend the length of the tube section, reducing the diameter of the tip section,
with the tip section in a reduced-diameter condition, inserting the applicator into a cavity of a patient to be treated,
removing the rigid wand from the applicator and causing the core to re-expand and to assume a fully-expanded, normal configuration within the patient, and
placing a radiation source within the lumen, inside the tip section, and irradiating tissue surrounding the tip section in accordance with a treatment plan.

27. The method of claim 26, wherein the tip section has a dome-shaped distal end, and the method including placing the radiation source in the lumen in a position relative to the dome-shaped end so as to emit radiation to produce a generally uniform isodose pattern around the distal end of the applicator.
